(19) United States
(12) Patent Application Publication
(10) Pub. No.: US 2006/0194207 A1
(43) Pub. Date: Aug. 31, 2006

(54) Kit for detecting nucleic acids

(75) Inventors: Yasumasa Mitani, Hiroshima-Ken (JP); Takanori Oka, Hiroshima-Ken (JP); Yoshihide Hayashizaki, Ibaraki-Ken (JP); Toshizo Hayashi, Tokyo-To (JP)

Correspondence Address: HELLER EHRMAN WHITE & MCAULIFFE LLP, 1717 RHODE ISLAND AVE, NW, WASHINGTON, DC, 20036-3001, US

(73) Assignees: Riken, Saitama-ken (JP); Kabushiki Kaisha Danform, Tokyo-to (JP); Wakunaga Pharmaceutical Co., Ltd., Osaka-fu, (JP)

(21) Appl. No.: 10/566,134

(22) PCT Filed: Jul. 29, 2004
(86) PCT No.: PCT/JP04/10851

§ 371 Date: Jan. 27, 2006

(30) Foreign Application Priority Data
Jul. 30, 2003 (JP) 2003-204015

(51) Int. Cl. C12Q 1/68; C12M 1/34

(52) U.S. Cl. 435/6; 435/287.6; 435/309.1; 435/288.2

(57) ABSTRACT

The present invention provides a reactor for carrying out in a single container the steps of the extraction of nucleic acids and the amplification of a nucleic acid, the procedures of the steps being usually different to each other. The reactor according to the present invention is a reactor for detecting a target nucleic acid from a sample, comprising at least a first compartment which contains an extraction reagent composition for extracting nucleic acids from said sample, a second compartment which contains an amplification reagent composition for amplifying the target nucleic acid, a separating means for separating the first and second compartments, and an aperture which enables to introduce said sample into only said first compartment. The separating means breaks the separation of the first and second compartments by physical energy supplied from the outside of the reactor, and thereby makes it possible to mix the extraction reagent composition in the first compartment and the amplification reagent composition in the second compartment.

### BACKGROUND OF THE INVENTION

1. Field of the Invention

The present invention relates to a reactor in which the extraction of nucleic acids from a sample and the amplification of a target nucleic acid can be performed, a kit for detecting nucleic acids comprising the reactor, and a process for detecting nucleic acids by using the reactor.

2. Background Art

Gene testing is effective as a diagnostic method of diseases or disorders, and a variety of techniques thereof have been put to clinical use. Many of these techniques utilize nucleic acid amplification methods such as polymerase chain reaction (PCR) and the like.

Clinical gene testing requires special biological procedures, which are usually performed with a plurality of containers or devices and often carried out in a plurality of areas within a laboratory. Thus, it is necessary in gene testing to transfer biological samples or reagents into other containers or to transport them to other areas. Accordingly, the contamination of the samples caused by the other clinical samples or amplification products as well as the contamination of the other samples by, for example, the scattering or aerosolization of the samples are acknowledged as a problem. Also, it is necessary to handle prudently a sample, since it is unknown what type of pathogen is contained in the sample. Moreover, it is necessary to carry out gene testing with special and expensive devices and equipments. In addition, when many samples are treated simultaneously, such treatment involves a risk of taking a wrong sample.

In particular, the contamination of the sample described above may bring a pseudo-positive result, and prevention thereof is an important problem to be solved. Particularly, in the gene testing involving the nucleic acid amplification method, the sample may be easily contaminated by amplification products (amplicons) of the preceding amplification reaction which has been conducted with the identical devices and apparatuses, and thus result in pseudo-positive result.

Several methods have been proposed in order to solve these problems. For instance, U.S. Pat. No. 2,675,989 discloses an apparatus for amplifying nucleic acids. In this apparatus, a sample is introduced into a reaction chamber and a reaction solution is removed by moving the introduced sample with an air suction/discharge means. This apparatus needs to use a special air suction/discharge means. Since the apparatus is not provided with a detection means of amplification products, a further process such as electrophoresis is required for the detection of the products.

U.S. Pat. No. 5,229,297 discloses a cuvette for the amplification and detection of gene, the cuvette including a pathway for interconnecting a sample, amplifying reagents and the waste compartment. The cuvette is composed of a roller which is a special apparatus for squeezing and pressurizing the sample to a certain direction so that a wall for isolating the sample and the detection reagent is broken, and a mixture thereof is expelled through the pathway into the detection port and finally the waste compartment. The cuvette also requires use of special and complex means and containers.

International Publication WO 95/11083 discloses a disposable reaction tube for the amplification assay of nucleic acids. The lid of this reaction tube is penetrable, so that pipetter is penetrated through the lid and thereby the sample is transferred to the detection port without opening the lid after the amplification reaction. The reaction tube prevents the scattering of the sample and the contamination of the other samples due to the generation of aerosol. Moreover, while it also lowers the risk of pseudo-positive result, it does not eliminate problematic factors such as the risk of the infection of a pathogen present in the sample, the complexity of operation, or the necessity of special apparatuses.

### SUMMARY OF THE INVENTION

The present inventors have found that it becomes possible to perform the extraction of nucleic acids from a sample and the amplification of a target nucleic acid in one reactor containing separately reagent groups which are required for each of the above steps. The present invention is based on this finding.

Accordingly, the object of the present invention is to provide a reactor for carrying out in a single container the steps of the extraction of nucleic acids and the amplification of a nucleic acid, the procedures of the steps being usually different to each other, a kit for detecting a nucleic acid comprising the reactor, and a process for detecting a nucleic acid by using the reactor.

The reactor according to the present invention is a reactor for detecting a target nucleic acid from a sample, comprising at least a first compartment which contains an extraction reagent composition for extracting nucleic acids from said sample, a second compartment which contains an amplification reagent composition for amplifying the target nucleic acid, a separating means for separating the first and second compartments, and an aperture which enables to introduce said sample into only said first compartment, wherein said separating means breaks the separation of the first and second compartments by physical energy supplied from the outside of the reactor, and thereby makes it possible to mix the extraction reagent composition in said first compartment and the amplification reagent composition in said second compartment.

The kit for detecting the nucleic acids according to the present invention comprises at least the reactor according to the present invention and a sampling device for collecting a sample.

The process for detecting nucleic acids according to the present invention is a process for detecting a target nucleic acid from a sample by using a reactor according to the present invention, comprising the steps of:

(a) bringing the sample into contact with the extraction reagent composition in said reactor and extracting nucleic acids in the sample;

(b) mixing a plurality of reagent compositions in the reactor by physical energy supplied from the outside of said reactor;

(c) conducting amplification reaction in said reactor; and

(d) detecting a signal from an amplification product.

According to the present invention, it is possible to carry out the extraction of nucleic acids from a sample and the amplification of a target nucleic acid in a single reactor. Therefore, the present invention enables decreasing the risks of the contamination of the sample due to the transfer of the reaction mixture into the other container and of the contamination of the other samples or environment. In addition, the reactor can be disposable, and thereby the risk of sample contamination due to the repeated use of the same container is eliminated. Further, according to the process for detecting nucleic acids of the present invention, no complicated biological procedures are required, and thereby even an unskilled person can detect the target nucleic acid rapidly and with high sensitivity.

### BRIEF DESCRIPTION OF THE DRAWINGS

FIG. 1 is a perspective illustration of a reactor including the extraction reagent composition and the amplification reagent composition and a sampling swab according to a preferred embodiment of the present invention.

FIG. 2 is a sectional view of the reactor and the sampling swab shown in FIG. 1, when the sample adhered to the tip of swab is in contact with the extraction reagent composition in the reactor.

FIG. 3 is a perspective illustration of a reactor including the extraction reagent composition, the pH adjusting reagent composition and the amplification reagent composition and a sampling swab according to a preferred embodiment of the present invention.

FIG. 4 is a diagrammatic scheme of the process for analyzing gene according to a preferred embodiment of the present invention.

FIG. 5 is an electrophoretogram for confirming the amplification of the target nucleic acid in the reactor according to the present invention.

### DESCRIPTION OF THE SYMBOLS

1. Lidding swab;
2. Stopper;
3. Tip of swab;
4. Reactor;
5. Stopper fitting part;
6. Membrane;
7. Extraction reagent composition;
8. Water-impermeable film;
9. Amplification reagent composition;
10. Water-impermeable film;
11. pH adjusting reagent composition;
401: Reactor according to the present invention;
402: Signal detecting apparatus;
403: Portable terminal;
404: Internet;
405: Computer for gene analysis;
406: Information storage device;
407: Information storage device.

### DETAILED DESCRIPTION OF THE INVENTION

A reactor according to the present invention comprises a first compartment containing an extraction reagent composition for extracting nucleic acids from a sample. A reagent(s) contained in the extraction reagent composition is not particularly limited and may be the one which enables a variety of methods for extracting nucleic acids known in the art. The constitution of the extraction reagent composition can be appropriately determined by persons skilled in the art on the basis of the methods used for extracting nucleic acids.

The methods for extracting nucleic acids are known such as the alkali extraction method, the phenol extraction method, the kaotropic reagent extraction method, the chromatographic purification method (WO 95/01359), and the ultracentrifugation method (Maniatis et al., 1982, Cloning: A Laboratory Manual, Cold Spring Harbor Laboratory, Cold Spring Harbor, N.Y.). Moreover, the methods for extracting nucleic acids by decomposing proteins in a sample with unspecific proteolytic enzymes such as proteinase K, protease or subtilisin is also known. In this connection, when the proteolytic enzyme is used, it is necessary to inactivate the enzyme prior to mixing the extraction reagent composition and the amplification reagent composition.

In a preferred embodiment of the present invention, the extraction reagent composition is a reagent composition for alkali extraction, preferably an aqueous sodium hydroxide solution. A pH value of the reagent composition for alkali extraction is preferably 8 or more, more preferably 11 or more, further more preferably 12 or more.

The reagent composition for alkali extraction may contain a surfactant(s). The surfactant may be any one of cationic, anionic, amphoteric, and nonionic surfactants. These surfactants include, but are not limited to, for example, cetyltrimethylammonium bromide (CTAB), sodium dodecyl sulfate (SDS), sodium N-lauroylsarcosinate, CHAPS (3-[(3-cholamidopropyl)-dimethylammonio]-1-propanesulfonate), polyoxyethylenesorbitanemonolaurate (Tween 20), and the like. The concentration of the surfactant in the composition is not particularly limited but preferably in the range of 0.005-5% (w/v), more preferably 0.01-2% (w/v).

As the method for extracting nucleic acids, it is also possible to utilize a method for extracting nucleic acids by decomposing or denaturing proteins and the other contaminants in a sample with a protein denaturing agent, and this method is particularly effective in the case of the extraction of RNA. The protein denaturing agent may be the one which can solubilize proteins, and includes for example kaotropic substances comprising guanidine salts such as guanidine hydrochloride, guanidine thiocyanate, or guanidine carbonate, or urea. Particularly, guanidine hydrochloride, guanidine thiocyanate, and the like are preferred. It is possible to suppress efficiently the action of RNase which may be contaminated in a biological sample by using the protein denaturing agent. Furthermore, chelating agents such as sodium citrate, EDTA, and the like, or reducing agents such as dithiothreitol (DTT), β-mercaptoethanol, and the like may be also used which may suppress the action of nucleic acid decomposing enzymes.

The reactor according to the present invention further comprises a second compartment containing the amplification reagent composition for amplifying the target nucleic acid. A reagent(s) contained in this amplification reagent composition is not particularly limited and may be the one which enables a variety of nucleic acid amplification methods. The constitution of the amplification reagent composition can be appropriately determined by persons skilled in the art depending on the nucleic acid amplification method used.

The nucleic acid amplification method may be the one which can amplify the target nucleic acid of interest from a solution containing nucleic acids (i.e. RNA or DNA) extracted from a sample, and various methods are known as such method (in general, see D. Kwoh and T. Kwoh, Am. Biotechnol. Lab. 8, 14-25, 1990). Appropriate nucleic acid amplification methods include, for example, the polymerase chain reaction method (PCR method; U.S. Pat. Nos. 4,683,195, 4,683,202, 4,800,159, and 4,965,188), the reverse transcriptase-PCR method (RT-PCR method; Trends in Biotechnology 10, pp 146-152, 1992), the ligase chain reaction method (LCR method; EP Laid-Open Publication No. 0320308, R. Weiss, Science 254, 1292, 1991), the strand displacement amplification method (SDA method; G. Walker et al., Proc. Natl. Acad. Sci. USA 89, 392-396, 1992; G. Walker et al., Nucleic Acids Res. 20, 1691-1696, 1992), the transcription-mediated amplification method (D. Kwoh et al., Proc. Natl. Acad. Sci. USA 86, 1173-1177, 1989), the self-sustained sequence replication (3SR; J. Guatelli et al., Proc. Natl. Acad. Sci. USA 87, 1874-1878, 1990), the Q beta replicase method (P. Lizardi et al., BioTechnology 6, 1197-1202, 1988), the nucleic acid sequence based amplification method (NASBA method; R. Lewis, Genetic Engineering News 12(9), 1, 1992), the repair chain reaction method (RCR method; R. Lewis, Genetic Engineering News 12(9), 1, 1992), the boomerang DNA amplification method (BDA method; R. Lewis, Genetic Engineering News 12(9), 1, 1992), the LAMP method (International Publication WO 00/28082), the ICAN method (International Publication WO 02/16639), and the like.

For instance, in the PCR method, a buffer solution containing heat-stable DNA polymerase, a pair of oligonucleotide primers designed based on the nucleotide sequences at the both ends of the target nucleic acid, dNTP, and the like is usually employed. Thus, in the case of utilizing the PCR method, the amplification reagent composition should include these reagents. In the PCR method, it is possible to amplify the target nucleic acid from DNA by repeated reactions consisting of the three steps of the dissociation of a double-stranded nucleic acid to single strands (denaturation), the annealing of a primer to the single stranded nucleic acid, and the synthesis of a complementary strand from the primer (elongation). In this method, the three steps in total in which the reaction solution is adjusted to each temperature suitable for the above three reactions are repeatedly conducted.

In the LCR method, two pairs of oligonucleotide probes are usually employed, in which the one pair binds to one strand of the target nucleic acid and the other pair binds to the other strand of the target nucleic acid. Each pair completely overlaps with the corresponding strand, respectively. The reaction is repeated in cycle until the sequence is amplified to a desired extent, in which the double-stranded nucleic acid in a nucleic acid sample is first denatured (that is, separated), the two pairs of oligonucleotide probes are next reacted with the strands in the presence of a heat-stable ligase, whereupon the oligonucleotide probes in each pair are ligated together, and the reaction product is separated. Thus, in the case of utilizing the LCR method, the amplification reagent composition contains the two pairs of oligonucleotide probes, the heat-stable ligase, the buffer solution, and the like described above.

In a preferred embodiment of the present invention, the amplification reagent composition is the one which enables amplification of the target nucleic acid under a constant temperature. Thus, the amplification reagent composition is the one which enables the isothermal amplification method, and the isothermal amplification method includes the 3SR method, the Q beta replicase method, the NASBA method, the SDA method, the LAMP method, the ICAN method described above, and the like. The preferred isothermal amplification method includes the SDA method, the LAMP method, and the ICAN method.

For instance, in the SDA method, the target nucleic acid can be amplified under the isothermal condition by using four primers in total, which involve a pair of amplification primers designed to include the recognition sites of the restriction enzymes and another pair of bumper primers between which are placed the amplification region. A nick is introduced in the restriction site on the amplification primers by the restriction enzyme, and elongation synthesis is conducted in the 3′-side of the amplification primers from the nick by DNA polymerase to displace the down-stream complementary strand of the target strand formed previously. This step is repeated without limit, because the restriction enzyme continuously introduces nicks into a complementary strand formed in the restriction site, and the DNA polymerase continuously forms a new complementary strand from the restriction site into which nicks have been introduced. Thus, in the case of utilizing the SDA method, the amplification reagent composition includes the four primers, the restriction enzyme, the DNA polymerase, the buffer, and the like.

As the isothermal amplification method, the amplification method with the isothermal amplification primers developed by the present inventors can be used preferably. This method employs a special primer (isothermal amplification primer) in the amplification method with use of the strand displacement reaction. The primer is a first primer comprising in its 3′-end portion a sequence (Ac′) which hybridizes a sequence (A) in the 3′-end portion of the target nucleic acid sequence in the first strand of a double-stranded template nucleic acid, and in the 5′-side of said sequence (Ac′) a sequence (B′) which hybridizes the complementary sequence (Bc) of a sequence (B) positioned in the 5′-side of said sequence (A) on the target nucleic acid sequence. In the absence of an intervening sequence between said sequences (Ac′) and (B′), (X−Y)/X is in the range of −1.00 to 1.00, in which X denotes the number of bases in said sequence (Ac′), and Y denotes the number of bases in the region flanked by said sequences (A) and (B) on the target nucleic acid sequence. In the presence of an intervening sequence between said sequences (Ac′) and (B′), {X−(Y−Y′)}/X is in the range of −1.00 to 1.00, in which X and Y have the same meanings as above, and Y′ denotes the number of bases in said intervening sequence. In this method, a second primer is also provided as a primer similarly designed for the other strand of said double-stranded template nucleic acid. The second primer comprises in its 3′-end portion a sequence (Cc′) which hybridizes a sequence (C) in the 3′-end portion of the target nucleic acid sequence in the second strand of the double-stranded template nucleic acid, and in the 5′-side of said sequence (Cc′) a sequence (D′) which hybridizes the complementary sequence (Dc) of a sequence (D) positioned in the 5′-side of said sequence (C) on said target nucleic acid sequence. In the absence of an intervening sequence between said sequences (Cc′) and (D′), (X−Y)/X is in the range of −1.00 to 1.00, in which X denotes the number of bases in said sequence (Cc′), and Y denotes the number of bases in the region flanked by said sequences (C) and (D) on the target nucleic acid sequence. In the presence of an intervening sequence between said sequences (Cc′) and (D′), {X−(Y−Y′)}/X is in the range of −1.00 to 1.00, in which X and Y have the same meanings as above, and Y′ denotes the number of bases in said intervening sequence. It is preferred to use these first and second primers as a primer pair.

In the absence of an intervening sequence between said sequences (Ac′) and (B′), said isothermal amplification primers are designed in such fashion that (X−Y)/X is in the range of −1.00 or more, preferably 0.00 or more, more preferably 0.05 or more, and even more preferably 0.10 or more, and in the range of 1.00 or less, preferably 0.75 or less, more preferably 0.50 or less, and even more preferably 0.25 or less. Moreover, (X+Y) is preferably in the range of 15 or more, more preferably 20 or more, even more preferably 30 or more, and preferably in the range of 50 or less, more preferably 48 or less, and even more preferably 42 or less.

In the presence of an intervening sequence between said sequences (Ac′) and (B′), the primer according to the invention is designed in such fashion that {X−(Y−Y′)}/X is in the range of −1.00 or more, preferably 0.00 or more, more preferably 0.05 or more, even more preferably 0.10 or more, and in the range of 1.00 or less, preferably 0.75 or less, more preferably 0.50 or less, and even more preferably 0.25 or less. Moreover, (X+Y+Y′) is preferably in the range of 15 or more, more preferably 20 or more, even more preferably 30 or more, and preferably in the range of 100 or less, more preferably 75 or less, and even more preferably 50 or less.

The above isothermal amplification primer is composed of deoxynucleotides and/or ribonucleotides, and has a strand length in which base pair bonding with the target nucleic acid can be conducted while required specificity is maintained under the given condition. The isothermal amplification primer has a strand length in the range of preferably 15-100 nucleotides, and more preferably 30-60 nucleotides. Also, the sequences (Ac′) and (B′) have the lengths preferably in the range of 5-50 nucleotides, and more preferably 10-30 nucleotides, respectively. If necessary, an intervening sequence having itself no influence on the reaction may be inserted between said sequences (Ac′) and (B′).

The DNA polymerases used in the isothermal amplification primer method provided by the present inventors may be those having strand displacement activities (strand displacement ability), and either of normal temperature, mesophilic or thermoduric polymerases may be successfully used. Also, the DNA polymerases may be either one of natural products or variants having been artificially varied. Furthermore, the DNA polymerases are preferably those having substantially no 5′->3′ exonuclease activities. Such DNA polymerases include, for example, a variant of a DNA polymerase derived from thermophilic bacillus bacteria such as Bacillus stearothermophilus (referred to hereinafter as B. st) and Bacillus caldotenax (referred to hereinafter as B. ca) of which the 5′->3′ exonuclease activity has been deleted, the Klenow fragment of an E. coli DNA polymerase 1, and the like.

The other reagents which may be used in the isothermal amplification primer method provided by the present inventors include catalysts such as magnesium chloride, magnesium acetate, magnesium sulfate, and the like; substrates such as dNTP mix, and the like; and buffers such as Tris-HCl buffer, Tricine buffer, phosphate Na buffer, phosphate K buffer, and the like. In addition, there may be used additives such as dimethyl sulfoxide, and betaine (N,N,N-trimethylglycine), acidic materials described in International Publication WO99/54455, cationic complexes, and the like.

According to the isothermal amplification primer method provided by the present inventors, it is possible to amplify nucleic acid sequences consisting of double-strand of interest. The principle consists in that said first and second primers are annealed to the first and second strands of the target nucleic acid (first and second template nucleic acids), respectively, to cause primer elongation reaction to synthesize first and second complementary nucleic acids containing the complementary sequences of said target nucleic acid sequences, respectively, and then the sequences (B′) and (D′) positioned in the 5′-side of the first and second complementary nucleic acids are hybridized with the sequences (Bc) and (Dc) on the same complementary nucleic acids, respectively, thereby allowing the portions of said sequences (A) and (C) on the first and second template nucleic acids to be single-stranded, respectively, and another primers having the same sequence as said primers are annealed to the single-stranded sequence (A) and (C) portions of the first and second template nucleic acids to conduct strand displacement reaction, thereby displacing the first and second complementary nucleic acids synthesized in the previous step by the complementary nucleic acids newly synthesized with said another primers.

In addition, only in the case that the isothermal amplification primer is annealed to the target nucleic acid to cause elongation reaction from the 3′-terminal side of the primer and the elongation product contains the target sequence, the isothermal amplification primer method provided by the present inventors enables the continuous amplification reaction by hybridizing the sequence of 5′-terminal of the primer to the elongation product, thereby enabling a similar isothermal amplification primer to anneal the hybridized elongation product and thus to realize continuous amplification reaction. On the other hand, when the isothermal amplification primer is annealed to any nucleic acids other than the target nucleic acid and elongation reaction is initiated from the 3′-terminal side of the primer, the elongation product does not contain the target sequence, and the sequence at the 5′-terminal of the primer cannot be hybridized to the elongation product and thus it becomes difficult to cause next annealing of a similar isothermal amplification primer, so that continuous amplification reaction become hard to occur and thus the amplification product is not obtained. Therefore, this amplification method exhibits higher specificity than the others. Moreover, since this method is a very specific amplification method, it is not always necessary to carry out operations of hybridizing the amplified product with DNA probes or the like and confirming whether the amplified product is the aimed amplified product or not.

The isothermal amplification primer method provided by the present inventors can be performed by keeping temperature at which the activity of an enzyme used can be maintained. Also, in order to anneal a primer to an target nucleic acid in the nucleic acid synthesis method according to the present invention, it is preferred, for example, to set up the reaction temperature at a temperature near the melting temperature (Tm) of the primer or less, and more preferred to set up the level of stringency in consideration of the melting temperature (Tm) of the primer. Therefore, the temperature is preferably in the range of 20° C.-80° C., more preferably in the range of about 35° C.-about 65° C.

The amplification reagent composition is set up for its pH so as to be suitable for amplification reaction on mixing with said extraction reagent composition. For instance, when a reagent composition for alkali extraction is used as the extraction reagent composition and a pH value thereof is excessively high for the amplification reaction, the amplification reagent composition is preferably adjusted preliminarily to a lower range of pH. The amplification reaction is appropriately conducted at pH generally in the range of 5-12, preferably 7-10.

In another embodiment of the present invention, the reactor according to the present invention comprises a third compartment containing a pH adjusting reagent composition for adapting the pH of said extraction reagent composition for the amplification reaction of the target nucleic acid with said amplification reagent composition, the third compartment being positioned between said first and second compartments. The pH adjusting reagent composition enables appropriate amplification reaction easily even if said extraction reagent composition is greatly different in pH from that suitable for the amplification reaction.

Acids which may be used for the pH adjusting reagent composition include mineral acids such as hydrochloric acid, sulfuric acid, nitric acid, phosphoric acid, and the like; carboxylic acids such as acetic acid, citric acid, futaric acid, fumaric acid, maleic acid, and the like; and organic sulfonic acids such as methanesulfonic acid, p-toluenesulfonic acid, and the like, and are preferably mineral acids, more preferably hydrochloric acid. Alkalis which may be used for the pH adjusting reagent composition include typically an aqueous sodium hydroxide solution.

The reactor according to the present invention further comprises separating means for separating the first and second compartments, and in the case of using the third compartment, separating means for separating the third compartment from the first and second compartments. The separating means breaks the separation between said first and second compartments by supplying physical energy from the exterior of the reactor, and thereby enables to mix the extraction reagent composition in said first compartment and the amplification reagent composition in said second compartment. When said third compartment is provided, the separations between said first compartment, said second compartment and said third compartment, and thereby enables to mix the extraction reagent composition in said first compartment, the amplification reagent composition in said second compartment, and the pH adjusting reagent composition in said third compartment. The methods for breaking the separations by physical energy include, for example, application of heat, irradiation of light, application of vibration or stress by machines or operators, or the like. The preferred separating means is the one which is breaked by applying heat.

The separating means may be impermeable at an ordinary temperature and a temperature used for the extraction of nucleic acids, and preferably includes, but not limited to, the means with water-impermeable films, more preferably the means with water-impermeable hydrophobic films. Also, it is preferred that such water-impermeable hydrophobic films can be molten by said physical energy from the exterior of the reactor. Moreover, the water-impermeable hydrophobic films preferably include the one which becomes a liquid having a density smaller than water. Thus, it is possible to prevent the leakage of the reagents from the reactors, as the water-impermeable hydrophobic film is again formed at the tops of the reagents after the amplification reaction in the reactor according to the present invention. Such water-impermeable hydrophobic films include, for example, waxes and their mixtures.

Wax is an organic material which forms a liquid having a density smaller than water upon melting by heating such as synthetic or natural waxes, e.g. those derived from minerals, plants or animals. In general, waxes are esters of high molecular weight fatty acids and high molecular weight alcohols. Typical pure waxes include eicosane (C₂₀H₄₂), octacosane (C₂₈H₅₈), cetyl palmitate (C₃₂H₆₄O₂), pentaerythritol tetrabehenate (C₉₃H₁₈₀O₈), and the like. Also, many useful wax mixtures are known as mixtures of materials which provide similar properties to those of pure waxes such as esters, fatty acids, high molecular weight alcohols, hydrocarbons, and the like. Such wax mixtures include, but not limited to, paraffines, PARAPLAST™ wax (Sherwood Medical), ULTRAFLEX™ wax (Petrolite Corporation), BESQUAR™ 175 wax (Petrolite Corporation), and the like. Waxes are commercially available or may be prepared by mixing with appropriate greases or oils which keep relative hardness, reduced adhesion specific for waxes and desirable melting temperature. It is also possible to mix the reagent compositions in the reactor according to the present invention in the desired combination by using paraffines having different melting temperatures to separate the reagents into two or three layers, which are molten, if necessary.

Said greases are organic materials, which remain solid or semi-solid at ordinary temperature (about 25° C.), becomes very soft at temperatures slightly lower than about 40° C., and is molten and turned into a liquid at a temperature of 40-80° C. Greases have densities smaller than water. Typical greases include white petrolatum such as vaseline, petroleum jelly, and the like, and it is a mixture of high molecular weight hydrocarbons. Said waxes are organic materials, which are solid at ordinary temperature (about 25° C.), but far harder than greases at temperatures lower than about 40° C., and melt and turn into a liquid having a density smaller than water at slightly higher temperatures. Waxes adheres to the surface of solids lesser than greases or oils.

It becomes possible to mix, if necessary, the reagent compositions which are respectively maintained separately in the reactor according to the present invention by using the separating means described above. In the preferred embodiment of the present invention, said water-impermeable film does not melt at a temperature for the extraction of nucleic acids with the extraction reagent composition, but melt at a temperature for the amplification of the target nucleic acid with said amplification reagent composition. Thus, it is possible to mix the respective reagent compositions by adjusting the temperature for amplification reaction after completing the extraction of nucleic acids.

In the reactor according to the present invention, the respective reagent compositions may be made solid. It is thus possible to prevent the leakage of the respective reagent compositions or the undesirable mutual mixing of the reagent compositions due to the vibration or the like during transportation or conveyance.

A method for fixing (solidifying) the reagent compositions may be the one which can fix a reagent dispersed uniformly within a reagent fixing layer, but is not limited specifically. Thus, as the fixing materials for fixing the reagents, there may be used materials for forming matrices, materials which do not form matrices, or combinations of these materials. Also, said fixing material may be the one which is dissolved or the one which is not dissolved together with the reaction reagents on the use of the reactor according to the present invention. In addition, as said fixing material, there are used fixing materials with which the fixed layer of the reagent composition obtained do not elute eluate components which impair the effect of the present invention.

In this connection, even if a material which forms a matrix and is not dissolved together with the reaction reagents is used as said fixing material, the molecule of the reaction reagent has a size far smaller as compared with the size of the matrix. Thus, if a solution containing a sample is brought into contact with the fixed reagent composition, the reaction reagent can be dissolved sufficiently into the solution.

The specific methods for fixing the reagent composition include, for example, a fixing method of the reagent composition by sealing it into a gel matrix such as agarose gel, alginic acid gel, carrageenan gel, curdlan gel, chitosan gel, or the like, a fixing method of the reagent composition by incorporating it into a light hardening resin such as light crosslinking polyvinyl alcohol, or a three-dimentional cross-linked structure such as polyacrylamide, a fixing method of the reagent composition with a water soluble viscous material such as CMC, and the like. Many of these fixing methods involve a step for adding the reagent compositions to the raw material of the fixing material and mixing them. In this connection, the reagent compositions may be added directly or in the form of a solution. Furthermore, it is also possible to fix the reagents by combining these methods. The definite fixing methods described above, in which the reagent compositions are fixed in a wet state, is advantageous to the case of using a reagent which should not be dried particularly.

In a preferred embodiment of the present invention, at least one of said extraction reagent composition and said amplification reagent composition is entrapped in a gel which can be molten by physical energy from the exterior of the reactor. When said pH adjusting reagent composition is contained in the reactor according to the present invention, the pH adjusting reagent composition may be entrapped in the gel. The method for melting the gel by physical energy includes application of heating, irradiation of light, and the like. Preferred gel is the one which is molten by heating. Moreover, said gel is preferably the one which is not molten at a temperature on the extraction of nucleic acids with said extraction reagent composition but should be molten at a temperature on the amplification of the target nucleic acid with said amplification reagent composition. It will thus be possible to mix the respective reagent compositions by adjusting temperature for the amplification reaction after the extraction of nucleic acids in the reactor according to the present invention.

The reagent compositions fixed by the method described above may be in a dry state. Thus, it is possible to prevent the denaturation of the reagent compositions due to storing for a long period. Drying methods include general techniques such as drying by heating, drying in vacuum, and lyophilization. It is also possible to utilize a stable drying technique of reagents described in U.S. Pat. No. 4,891,319 and a method for stabilizing an enzyme for nucleic acid amplification described in Japanese Patent Publication No. 10-503383. Moreover, it is possible to use trehalose and/or polyvinylpyrrolidone as a stabilizing agent, and thereby enzymes such as DNA polymerase can be stably maintained particularly in the case of the lyophilization of the reagents. When the fixed reagent composition has been dried, a fluid such as water may be supplied, if necessary, in the use of the reactor according to the present invention.

The reactor according to the present invention further comprises an aperture which enables to introduce said sample into said first compartment. Thus, the sample introduced from the aperture of the reactor is contacted only with the extraction reagent composition without contact with other reagent compositions, and it is possible to extract nucleic acids efficiently.

In the reactor according to the present invention, the extraction reagent composition and the amplification reagent composition are separately retained, and these compositions are mixed after the extraction of nucleic acids and before the nucleic acid amplification reaction. The mixing of these reagent compositions may be conducted by general methods such as the convection of a fluid by heating, vibration of the reactor, or the like, and a mixing means may be incorporated into the reactor in order to conduct the mixing more efficiently. Such mixing means includes, for example, the preliminary charge of a carrier such as beads in the reactor. Particularly, in the upper portion of the reactor is provided the water-impermeable film, in which the carrier is fixed, the film is molten by physical energy such as heat, and the carrier is dropped into the lower portion thus resulting in the mixing of the reagent compositions. Also, said carrier can be formed in a shape of propeller with use of nanotechnology to improve the efficiency of mixing by the rotation of the propeller during the drop from the upper portion to the lower portion within the reactor. In addition, magnetic beads may be used as said carrier, and in that case the reagent compositions can be mixed by moving the magnetic beads in the reactor by exterior magnetism. Moreover, as the other mixing means, the above described water-impermeable film is preliminarily fixed in the lower portion of the reactor, and in that case the film is molten by physical energy such as heat and thus the molten film is moved to the upper portion, so that the reagent compositions can be mixed. Therefore, the reactor according to the present invention has preferably a part of which inner cross-sectional area decreases in the direction from the aperture to the bottom. The molten film becomes easily movable to the upper portion by forming the reactor in such shape.

While the target nucleic acid amplified with the reactor according to the present invention may be detected by general methods such as the method with use of a specific probe to which a detectable label is attached, it is also possible to constitute the reactor so that signals based on the presence of the amplification product can be generated. Therefore, according to the preferred embodiment of the present invention, the reactor according to the present invention further comprises a signal generating means based on the amplification product. Also, in that case the reactor according to the present invention is preferably the one to which the signal from the nucleic acid amplification product is permeable. Thus, it is possible to detect the signal without removing the amplification product from the reactor.

As the signal generating means, the means which are known to persons skilled in the art can be used, and include, but not limited to, for example, intercalators such as ethidium bromide, SYBR Green I (Molecular Probe), and the like. These intercalators link to a double-stranded DNA, and thus the fluorescent intensity and the concentration of the double-stranded DNA are directly proportional to each other. Thus, an intense fluorescence of the intercalator shows the presence of a high concentration of the amplification product, and thereby the target nucleic acid is detected. Therefore, it is possible to generate signals based on the amplification product by preliminarily incorporating such intercalator into the amplification reagent composition. Also, there may be used as the signal generating means Fluorescence Resonance Energy Transfer (FRET), or the like. FRET occurs only when two probes in close vicinity to each other are hybridized to the amplification product, but it does not occur in the absence of a specific DNA to which hybridization probes adjacent to each other can be hybridized. Thus, two probes which can be hybridized specifically to each of the two regions close to each other in the target nucleic acid may be incorporated preliminarily into the amplification reagent composition. Also, in the course of nucleic acid amplification, pyrophosphoric acid ion is liberated from a substrate (dNTPs) and linked with magnesium ion in the amplification reagent composition to produce magnesium pyrophosphate, which clouds the reaction solution. It is possible to judge visibly the presence of the amplification product. It is also possible to detect the amplification product by intercalating an intercalator into the amplification product, transmitting an electric current to the amplification product, and recording the difference of the electric currents or voltages. Moreover, a primer may be preliminarily linked with a carrier such as beads or gold colloid particles. In this case, said carrier is agglomerated by the amplification of the target nucleic acid, and it is possible to detect the amplification product by visibly confirming the agglomeration.

The signal permeable reactor can be selected easily by persons skilled in the art depending on its signal generating means. For instance, when the signals are visibly detected, the materials of the reactor are transparent or translucent and can be made of a thermoplastic polymer such as plastics. As the thermoplastic polymers, it is possible to use polypropylene, polystyrene, polymethylpentene, and copolymers or mixtures thereof. Alternately, it may be used a transparent glass. Also, in the case of reading fluorescent signals, chromogenic signals, luminescent signals, it is preferred to use the ones made of the similar materials. Moreover, when ultraviolet light or infrared radiation is utilized, the materials permeable to these radiations are preferably used. In addition, it is possible to use electrochemical measurement in order to detect the nucleic acid amplification product in the reaction solution or the by-products of the nucleic acid amplification reaction, and in such cases containers such as a container made of a material which transmits electric current, e.g., a carbon-coated container are preferably used.

The reactor according to the present invention can constitute a kit for detecting nucleic acids in combination with the other devices used for the amplification of the target nucleic acid from a sample. The other devices include typically a sampling device. Thus, another aspect of the present invention provides a kit for detecting nucleic acids which comprises at least the reactor according to the present invention and a sampling device for collecting a sample.

The sampling devices include ordinary devices such as pipette or syringe for suctioning fluids, or spatula. In addition, samples such as human buccal mucosal cells and sputum can be sampled easily with a swab.

The swab may be the ordinary one which comprises a sampling part made of a cotton material and a handling part made of a material such as a polystyrene tube material, and the swab which can cap the reactor according to the present invention by inserting the swab into the aperture of the reactor is preferred. Also, the swab is preferably the one which closes the opening between it and the reactor by inserting it into the aperture of the reactor. Thus, in a preferred embodiment of the present invention, said swab can bring the collected sample into contact with the extraction reagent composition in the reactor according to the present invention, and can seal the aperture of said reactor. The swab is more preferably the one which has been processed with a thermoplastic polymer such as plastic. As the thermoplastic polymer there can be used polypropylene, polystyrene, polymethylpentene, and copolymers or mixtures thereof. Moreover, a swab used in the case of simultaneous examination of a large number of samples can be separated at the slender part of the handle into two parts, and furthermore it is possible to distinguish a sample in the reactor by recording the same reference number at both ends and storing the sample. The swab preferably has an extrusion at the tip of the sample collecting part so as the sample to be collected in a larger amount.

In a preferred embodiment of the present invention, the kit according to the present invention further comprises a detachment preventing means for preventing the detachment of said swab from said reactor. Thus, after the collection part has been once inserted into the reactor according to the present invention, it is possible to prevent the unintended detachment of the swab from the reactor. Said detachment preventing means may comprise a convex or concave portion provided on said swab and the corresponding concave or convex portion provided on said reactor.

Alternately, said swab may be the one which is bonded to the lid of the reactor, so that simultaneously with the contact of the sampling part of the swab with the extraction reagent composition in the reactor according to the present invention the reactor is sealed tightly by the lid.

The kit according to the present invention may comprise a lid to close the aperture of the reactor apart from the lid of said swab. This lid need not be necessarily a firm one, but may be any of lids which prevent the contamination of an alien substance. Such a lid may include a cellophane tape, a laminate, a wrap, or a seal which prevents the contamination of alien substances at most, or a water-impermeable film such as wax for covering the reactor.

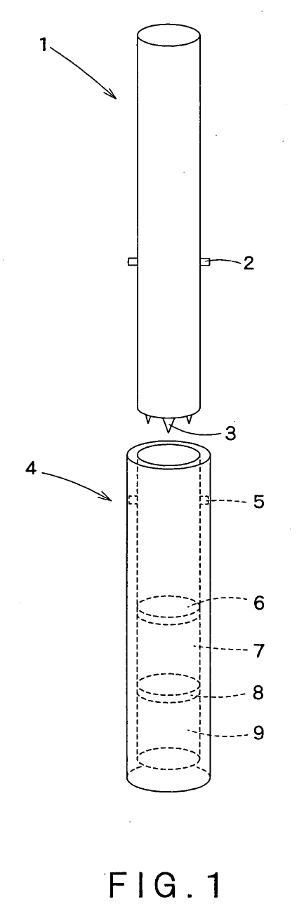

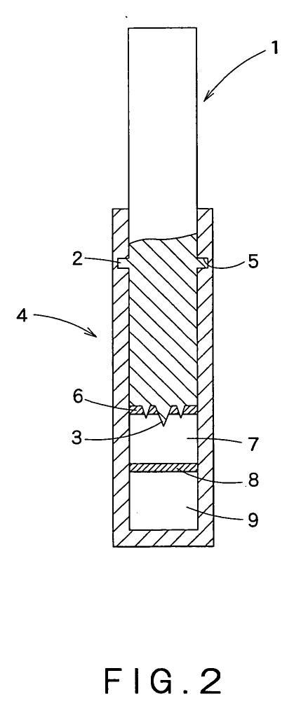

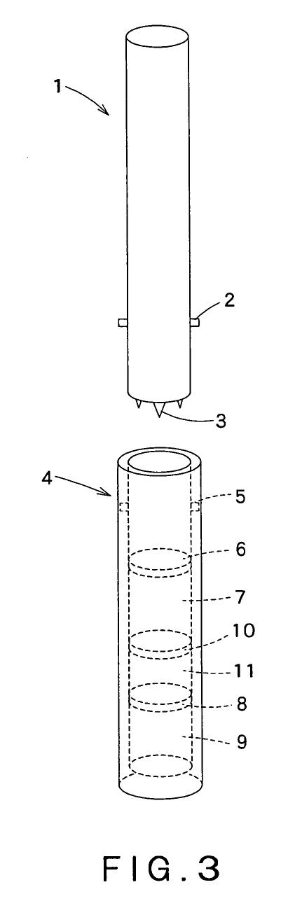

The combinations of the reactor and the swab according to the preferred embodiment of the present invention are shown in FIGS. 1-3. A swab 1 which also serves as a lid shown in FIG. 1 has a swab tip 3 provided with extrusions at its lowest end, which can collect efficiently a sample. The swab 1 which also serves as a lid is further provided with a stopper 2. On the one hand, the reactor 4 contains the extraction reagent composition 7 and the amplification reagent composition 9 which are separated by a water-impermeable film 8. Also, the reactor 4 has an inside diameter almost equal to the outside diameter of the swab which also serves as a lid 1, and is further provided with a stopper fitting part 5 which can fit with a stopper 2. The upper surface of the extraction reagent composition 7 is covered with a film 6 which prevents the contamination of alien substances and the leak of reagents. FIG. 2 illustrates the sectional view in which a sample is collected with said swab which also serves as a lid 1 and applied to said reactor 4. In FIG. 2, the extrusion at the tip 3 of the swab breaks through the film 6, and thereby the sample attached to the protrusion at the tip 3 of the swab is contacted with the extraction reagent composition 7. Also, the reactor 4 is sealed tightly with the swab which also serves as a lid 1 and has about the same diameter as the inside diameter of the reactor, and a stopper 2 and a stopper fitting part 5 maintain its state. In this state, nucleic acid extraction is conducted, a water-impermeable film 8 is molten by applying physical energy such as heat to the reactor 4, the extraction reagent composition 7 and the amplification reagent composition 9 are thereby mixed, the reactor 4 sealed tightly by the swab which also serves as a lid 1 is then set at a temperature lower than a certain temperature thus enabling to amplify the target nucleic acid. FIG. 3 illustrates the reactor 4 which comprises a pH adjusting reagent composition 11 between the extraction reagent composition 7 and the amplification reagent composition 9, these compositions being separated by the water-impermeable films 8 and 10, and the swab which also serves as a lid 1.

The reactor according to the present invention is used for the detection of the target nucleic acid from a sample. Thus, another aspect of the present invention provides a process for detecting a target nucleic acid from a sample by using a reactor according to the present invention, comprising the steps of:

(a) bringing the sample into contact with the extraction reagent composition in said reactor and extracting nucleic acids in the sample;

(b) mixing a plurality of reagent compositions in the reactor by physical energy supplied from the outside of said reactor;

(c) conducting amplification reaction in said reactor; and

(d) detecting a signal from an amplification product.

The sample may be the one which is suspected of containing the target nucleic acid without particular limitation, and includes, for example, samples derived from living things, processed foods, drainage, drinking water, air, and the like. Also, the living things may be any one of animals, plants or microbes. Moreover, the animals are preferably mammals, and more preferably human beings. Samples from animals include blood, feces, phlegm, mucus, serum, urine, saliva, tear, biopsies, histological tissue samples, tissue cultures, and the like. Also, samples from plants include agricultural products, foliage plants, natural edible plants, and the like.

The target nucleic acid may be the one from which useful information is obtained by the detection thereof, and include, but not limited to, for example the ones having nucleic acid sequence specific to a wild type gene, a mutated gene or a pathogen. The pathogen includes, for example, virus, bacteria, fungi, and the like. For instance, when a wild type gene is aimed as the target nucleic acid, diseases caused by the deficiency of the gene are detected by the undetectability of this nucleic acid. Also, when a mutated gene is set as the target nucleic acid, diseases caused by the genetic mutation is detected by the detection of this nucleic acid. Moreover, when a nucleic acid having a sequence specific to a pathogen is set as the target nucleic acid, infections caused by the pathogen are detected by the detection of this nucleic acid.

Each steps described above in the nucleic acid detecting method according to the present invention can be performed depending on the constitutions of the reactor according to the present invention such as the nucleic acid extracting method employed in said reactor, the separation means between the respective reagent compositions, and the nucleic acid amplification methods. In addition, it can be appropriately carried out by persons skilled in the art to detect signals from a nucleic acid amplification product by general methods such as a method with use of a specific probe to which a detectable label is attached. Also, when a signal generating means is preliminarily provided in the reactor, signals can be detected easily with this means.

As a means for the useful application of the results of the nucleic acid detecting method according to the present invention, it is also possible to input signals detected in the above described step (d) or results obtained by the signals into a computer for genetic analysis and to output the analytical results by the computer. Therefore, the present invention provides a process of analyzing a gene, comprising, following the above described steps (a)-(d):

(e) inputting the detected signal into the computer for genetic analysis;

(f) in said computer, comparing said signal with information available to the computer, and thereby conducting the characterization of said signal and/or the search of information related to said signal; and

(g) outputting from said computer the characteristics of said signal and/or the information related to said signal. The input into the computer in the above described step (e) and the output from the computer in the above described step (g) are performed preferably through a communications network such as internet.

According to the genetic analysis process according to the present invention, it is possible to obtain more detailed information for example by connecting a signal detection apparatus to a communication apparatus, sending the signals obtained to a genetic analysis center or the like to conduct more detailed analysis, and receiving the detailed analytical results and the related information. As the communication apparatus, there are preferably used a personal computer, a portable terminal such as portable telephone, and the like which can send or receive information through a communication network such as internet.

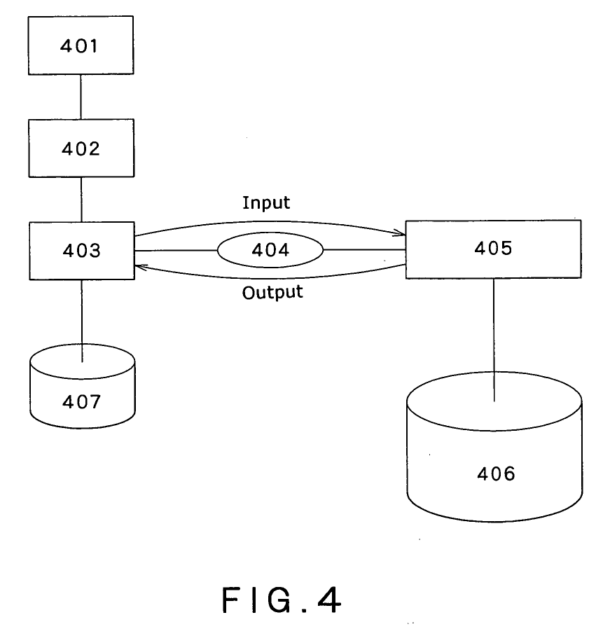

The schematic illustration of the genetic analysis according to the preferred embodiment of the present invention is shown in FIG. 4. After the amplification reaction of the target nucleic acid is conducted in the reactor 401 according to the present invention, signals based on amplification products are detected by a signal detection apparatus 402. The detected signals are inputted into a computer for gene analysis 405 through internet 404 by a portable terminal 403. In this computer for gene analysis 405, the inputted signals, and information shown by the presence or absence of the target nucleic acid memorized in an information storage device 406 and information related thereto are compared, and thereby the characterization of the signal and/or the search of the signal related information are conducted. Next, the characteristics of said signal and/or the information related to said signal are outputted from the computer for gene analysis 405 through internet 404 into the portable terminal 403. The outputted information is stored in an information storage device 407.

The process for genetic analysis according to the present invention is, for example, a process for detecting diseases or disorders if the target nucleic acid indicates diseases or disorders by its presence or absence, and furthermore a process for obtaining information relating to the diseases or disorders. In this case, the outputted characteristics of signal include the names of the diseases or disorders indicated by said signal, the names of genes containing the target nucleic acid, and the like, and the outputted relating information include the instructions of the diseases or disorders, the countermeasures against the diseases or disorders, the methods for further precise diagnosis, and the like. Particularly, complicated analysis can be conducted in the computer for genetic analysis, so that when a plurality of genes participating in the aimed diseases or disorders are present, it is possible to obtain more correct analytical results by conducting the nucleic acid detection methods on the respective genes and sending all of the results (signals).

Particularly, the nucleic acid detection method according to the present invention can be performed by a subject himself, and the leak of genetic information is prevented by conducting also genetic analysis with a communication means by the subject himself. Moreover, it is possible to maintain or control complicated genetic information by controlling personal information with use of a private portable terminal. It is also possible to select a hospital or drugstore depending on a personal object on the basis of the genetic information.

Moreover, the reactor according to the present invention can be used for the diagnosis or the judgment of development risk of diseases or disorders as described above. Therefore, another aspect of the present invention provides a use of the reactor according to the present invention in the diagnosis or the judgment of development risk of diseases or disorders.

### EXAMPLES

The present invention is more particularly described with reference to examples, but the scope of the present invention is not limited to these examples.

### Example 1

In this example, the partial sequence (SEQ ID NO. 1) of human STS DYS237 contained in human genome was amplified and detected. And, as a primer there was used the following primer pair which makes capable of amplification by isothermal reaction at 60° C.: SY153LP 13-15: 5′-CATTTGTTCAGTAG CATCCTCATTTTATGTGCA-3′ (SEQ ID NO. 2: the underlined part corresponds to the first-twentieth nucleotide in SEQ ID NO. 1, and another part corresponds to the complementary sequence of the thirty-sixth-forty-eighth nucleotide), and SY153RP13-15: 5′-CTTGCAGCATCACCAACCCAAAAGCACTGAGTA-3′ (SEQ ID NO. 3: the underlined part corresponds to the complementary sequence of the 120th-139th nucleotide in SEQ ID NO. 1, and another part corresponds to the 92nd-104th nucleotide).

A kit for detecting nucleic acid was prepared as follows. First, an amplification reagent (in 20 p1 in total, containing 20 mM Tris-HCl (pH 8.8), 10 mM KCl, 10 mM (NH₄)₂SO₄, 2 mM MgSO₄, 1% Triton X-100, 4 mM dNTP, 100 pmol of the respective primers, 8 U Bst DNA polymerase (New England Biolabs), and SYBR®-Green I (Molecular Probes) diluted to 10,000 folds) was added to the bottom of a transparent cylindrical reactor (inner diameter: 2-3 mm) (amplification reagent layer). A 10 μl portion of a heat molten liquid paraffin (Kanto Chemical Co., Ltd.: melted at 50-52° C.) was layered on the amplification reagent layer (hydrophobic film layer). After the paraffin was solidified, 0.01 N NaOH (5 μl) was layered on the solidified layer as a pretreatment reagent (pretreatment reagent layer).

Next, the kit for detecting nucleic acid was used to detect a target sequence from buccal mucosal cells of a human subject. First of all, the human buccal mucosal cells were collected with a swab and added to the pretreatment reagent layer portion. The buccal mucosal cells were left standing at room temperature for 30 minutes for denaturation, and human genomic DNA was extracted. Next, the reactor was maintained at 60° C. for 1 hour to conduct amplification reaction. The hydrophobic film layer was molten by maintaining the reactor at 60° C. and moved to the top of the reactor, and thereby the pretreatment reagent layer and the amplification reagent layer have been mixed. The same experiment was also carried out for a sample to which human mucosal cells were not added. Finally, UV (245 nm) was irradiated to detect the amplified aimed nucleic acid.

In the reactor to which human mucosal cells were added, fluorescent signals caused by SYBR®-Green I were visibly confirmed. On the other hand, no fluorescent signals were confirmed in the reactor to which human mucosal cells were not added. It was indicated from these results that the extraction of human genomic DNA, the amplification of the target sequence and the detection of an amplified product are possible by using the kit for detecting the nucleic acid.

### Example 2

In this example, target sequences for amplification and detection and the primer pair used were the same as those in Example 1.

A kit for detecting nucleic acid was prepared as follows. First, an amplification reagent (in 20 μl in total, containing 20 mM Tris-HCl (pH 8.8), 10 mM KCl, 10 mM (NH₄)₂SO₄, 8 mM MgSO₄, 0.1% Tween 20, 0.1% Triton X-100, 1.4 mM dNTP, 0.8% DMSO, 1600 pmol of the respective primers, and 16 U Bst DNA polymerase (New England Biolabs)) was added to the bottom of a transparent cylindrical reactor (inner diameter: 2-3 mm) (amplification reagent layer). A 10 μl portion of a heat molten liquid paraffin (Kanto Chemical Co., Ltd.: melted at 50-52° C.) was layered on the amplification reagent layer (hydrophobic film layer). After the paraffin was solidified, 0.01 N NaOH (5 μl) was layered on the solidified layer as a pretreatment reagent (pretreatment reagent layer).

Next, the kit for detecting nucleic acid was used to detect a target sequence from buccal mucosal cells of a human subject. First of all, the human buccal mucosal cells were collected with a swab and added to the pretreatment reagent layer portion. The buccal mucosal cells were left standing at room temperature for 30 minutes for denaturation, and human genomic DNA was extracted. Next, the reactor was maintained at 60° C. for 1 hour to conduct amplification reaction. The hydrophobic film layer was molten by maintaining the reactor at 60° C. and moved to the top of the reactor, and thereby the pretreatment reagent layer and the amplification reagent layer have been mixed. The same experiment was also carried out for a sample to which human mucosal cells were not added.

In the reactor to which human mucosal cells were added, the cloudiness of the reaction solution due to magnesium pyrophosphate produced by the amplification reaction was confirmed. On the other hand, no cloudiness was confirmed in the reactor to which human mucosal cells were not added. It was indicated from these results that the extraction of human genomic DNA, the amplification of the target sequence and the detection of an amplified product are possible by using the kit for detecting the nucleic acid.

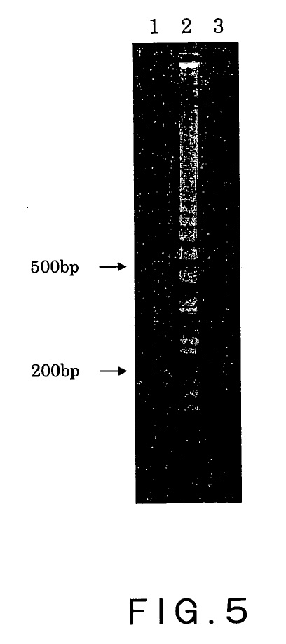

Furthermore, a 5 μl portion of the reaction solution after amplification reaction was subjected to electrophoresis in 3% NuSieve 3:1 Agarose gel (TaKaRa Shuzo Co., Ltd.). An electrophoretic photogram representing the results is shown in FIG. 5. In FIG. 5, Lane 1 represents a 20 bp DNA Ladder size marker, Lane 2 represents a sample to which the human buccal mucosal cells have been added, and Lane 3 represents a control sample to which the human buccal mucosal cells have not been added. In the control sample (Lane 3), no bands were observed except that the unreacted primer was stained. In the sample to which the human buccal mucosal cells have been added (Lane 2), a band in the vicinity of about 160 bp expected as the aimed amplification product was confirmed. It has been verified from these results that the extraction of human genomic DNA, the amplification of the target sequence and the detection of an amplified product are possible by using the kit for detecting the nucleic acid.

1. A reactor for detecting a target nucleic acid from a sample, comprising at least a first compartment which contains an extraction reagent composition for extracting nucleic acids from said sample, a second compartment which contains an amplification reagent composition for amplifying the target nucleic acid, a separating means for separating the first and second compartments, and an aperture which enables to introduce said sample into only said first compartment,
wherein said separating means breaks the separation of the first and second compartments by physical energy supplied from the outside of the reactor, and thereby makes it possible to mix the extraction reagent composition in said first compartment and the amplification reagent composition in said second compartment.

2. The reactor according to claim 1, wherein said separating means comprises a water-impermeable film which can be molten by physical energy from the outside of the reactor.

3. The reactor according to claim 2, wherein said water-impermeable film is not molten at a temperature for the extraction of nucleic acids with said extraction reagent composition, whereas the film is molten at a temperature for the amplification of the target nucleic acid with said amplification reagent composition.

4. The reactor according to claim 1, wherein at least one of said extraction reagent composition and said amplification reagent composition are entrapped in a gel which can be molten by physical energy from the outside of the reactor.

5. The reactor according to claim 4, wherein said gel is not molten at a temperature for the extraction of nucleic acids with said extraction reagent composition, whereas the gel is molten at a temperature for the amplification of the target nucleic acid with said amplification reagent composition.

6. The reactor according to claim 1, further comprising a third compartment containing a pH adjusting reagent composition for adapting the pH of said extraction reagent composition for the amplification reaction of the target nucleic acid with said amplification reagent composition, the third compartment being positioned between said first and second compartments, and separating means for separating the third compartment and said first and second compartments,
wherein said separating means breaks the separation between said first, second and third compartments by physical energy supplied from the outside of the reactor, and thereby makes it possible to mix the extraction reagent composition in said first compartment, the amplification reagent composition in said second compartment, and the pH adjusting reagent composition in said third compartment.

7. The reactor according to claim 6, wherein said separation means comprises a water-impermeable film which can be molten by physical energy from the outside of the reactor.

8. The reactor according to claim 7, wherein said water-impermeable film is not molten at a temperature for the extraction of nucleic acids with said extraction reagent composition, whereas the film is molten at a temperature for the amplification of the target nucleic acid with said amplification reagent composition.

9. The reactor according to claim 6, wherein said pH adjusting reagent composition is entrapped in a gel which can be molten by physical energy from the outside of the reactor.

10. The reactor according to claim 9, wherein said gel is not molten at a temperature for the extraction of nucleic acids with said extraction reagent composition, whereas the gel is molten at a temperature for the amplification of the target nucleic acid with said amplification reagent composition.

11. The reactor according to claim 1, wherein said extraction reagent composition is a reagent composition for alkali extraction.

12. The reactor according to claim 1, wherein said amplification reagent composition enables amplification of a target nucleic acid under a constant temperature.

13. The reactor according to claim 1, wherein said reactor is permeable to signals from an amplification product.

14. The reactor according to claim 1, wherein said reactor has a part of which inner cross-sectional area decreases in the direction from the aperture to the bottom.

15. A kit for detecting nucleic acid, comprising a reactor according to any one of claim 1 and a sampling device for collecting a sample.

16. The kit according to claim 15, wherein said sampling device is a swab.

17. The kit according to claim 16, wherein said swab can bring the collected sample into contact with the extraction reagent composition in said reactor and can seal the aperture of said reactor.

18. The kit according to claim 17, further comprising a detachment preventing means for preventing the detachment of said swab from said reactor.

19. The kit according to claim 18, wherein said detachment preventing means comprises a convex or concave portion provided on said swab and the corresponding concave or convex portion provided on said reactor.

20. A process for detecting a target nucleic acid from a sample by using a reactor according to any one of claim 1, comprising the steps of:
(a) bringing the sample into contact with the extraction reagent composition in said reactor and extracting nucleic acids in the sample;
(b) mixing a plurality of reagent compositions in the reactor by physical energy supplied from the outside of said reactor;
(c) conducting amplification reaction in said reactor; and
(d) detecting a signal from an amplification product.

21. The process according to claim 20, wherein said target nucleic acid has a nucleic acid sequence specific to a wild type gene, a mutated gene or a pathogen.

22. The process according to claim 21, wherein said pathogen is virus, bacteria, or fungi.

23. A process of analyzing a gene by using a reactor according to any one of claim 1, comprising the steps of:
(a) bringing the sample into contact with the extraction reagent composition in said reactor and extracting nucleic acids in the sample;
(b) mixing a plurality of reagent compositions in the reactor by physical energy supplied from the outside of said reactor;
(c) conducting amplification reaction in said reactor;
(d) detecting a signal from an amplification product;
(e) inputting the detected signal into the computer for genetic analysis;
(f) in said computer, comparing said signal with information available to the computer, and thereby conducting the characterization of said signal and/or the search of information related to said signal; and
(g) outputting from said computer the characteristics of said signal and/or the information related to said signal.

24. The process according to claim 23, wherein the input into the computer in the step (e) and the output from the computer in the step (g) are performed through a communications network.

25. A use of the reactor according to any one of claim 1 in the diagnosis or the judgment of development risk of diseases or disorders.
